(19) United States
(12) Patent Application Publication
(10) Pub. No.: US 2024/0108266 A1
(43) Pub. Date: Apr. 4, 2024

(54) UROFLOWMETRY SYSTEM

(71) Applicant: CREATIVE MEDICAL SOLUTIONS INC., Toronto (CA)

(72) Inventors: Brian CARRILLO, Toronto (CA); Monica FARCAS, Toronto (CA); Tian Ge LI, Toronto (CA)

(21) Appl. No.: 18/472,462

(22) Filed: Sep. 22, 2023

Related U.S. Application Data
(60) Provisional application No. 63/409,918, filed on Sep. 26, 2022.

(51) Int. Cl. A61B 5/20; A61B 5/00

(52) U.S. Cl. CPC A61B 5/208; A61B 5/6887

(57) ABSTRACT

A uroflowmetry system comprising: a receptacle for receiving urine; a plumbing unit for directing urine from the receptacle; flow-through fluid sensors for measuring urine flow; an output port from which urine exits the system; a data acquisition unit for receiving and collecting urine flow data from the flow-through fluid sensors; and a data processing and storage unit for processing and storing urine flow data received from the microcontroller.

### CROSS REFERENCE TO RELATED INVENTION

This application claims benefit to U.S. Provisional Application No. 63/409,918, filed Sep. 26, 2022, the entirety of which is incorporated herein by reference.

### FIELD OF THE INVENTION

The present invention relates to urology, and in particular, to a uroflometry system for screening and diagnosing patients for conditions involving the urinary tract.

### BACKGROUND ART

Uroflowmetry systems can measure the volume of urine released from the body, the rate with which it is released, and how long the release takes. Such uroflowmetry systems are used to screen and diagnose patients for conditions involving the lower urinary tract. For example, they can help to determine if the patient has an obstruction or normal urine flow. Furthermore, uroflowmetry systems can be used as part of post-surgery or post-treatment evaluations which help a physician determine a patient's recovery progress.

An early example is taught in U.S. Pat. No. 2,648,981, where an apparatus comprises a receptacle which receives a flowing stream of urine from the patient, and this receptacle has a plurality of vertically-spaced outlet orifices from which the urine can exit the receptacle. Each of the vertically-spaced outlet orifices has a corresponding liquid-receiving compartment into which that particular outlet orifice discharges urine. When the patient's rate of flow of urine is higher, the urine reaches and exits from more outlet orifices, and the urine will collect in more liquid-receiving compartments. When the patient's rate of flow of urine is lower, the urine reaches and exits from fewer outlet orifices, and the urine will collect in fewer liquid-receiving compartments.

Numerous techniques and related systems for uroflowmetry have been more recently described, including spinning disc, capacitive, acoustic, magnetic induction, cantilever, float sensor, optical and paddlewheel turbine systems. However, these techniques and related systems are seldom employed in clinical practice.

A majority of current uroflowmetry systems operate using gravimetric techniques, where the patient's urine is collected in a receptacle that is placed upon a weight sensor, which records the weight of the urine at set time intervals.

Conventional gravimetric systems generally comprise the following basic components:
(i) a urine collection receptacle;
(ii) a weight sensing unit; and
(iii) an output unit (e.g. printer).

During a patient void, the receptacle fills with urine. The weight sensor continuously samples total weight of the urine at set time intervals. The final Weight vs. Time graph, and its derivative Flowrate vs. Time graph, plus additional clinical metrics (e.g. average and maximum flowrates, total volume voided), are subsequently output for clinician interpretation.

An example of a conventional gravimetric uroflowmetric system is taught by WO/2007/111001. A container for receiving urine is removably fixed on a mounting table. As the patient's urine is being received in the container, a measuring unit measures the weight of urine received by the container a plurality of times at predetermined time intervals. The system also has an output unit for outputting the measurements taken by the measuring unit.

Current uroflowmetry systems have multiple disadvantages which can broadly be categorized into:
(1) clinician workflow inefficiencies;
(2) patient discomfort; and
(3) lack of data reliability.

### 1. Clinician Workflow Inefficiencies

The urine collection receptacle must be manually emptied and cleaned after every use. This can be unsanitary and messy, and additionally adds an unavoidable interruption to workflow during already busy clinic hours.

Many urology clinics employ nursing staff specifically for the purpose of administering uroflow testing in order to avoid this interruption for physicians. This adds to the clinic's operating expenses.

### 2. Patient Discomfort

Any interruptions and inefficiencies to clinician workflow or delays in receptacle emptying and cleaning necessarily evolve into long wait times for patients in queue—sometimes up to hours. This is especially evident when a clinician has just begun an appointment with another patient—meaning the system may not be cleaned for 15-30 minutes if there is no dedicated uroflowmetry staff. The uroflowmetry system is therefore out-of-commission for this length of time.

Long waits can be frustrating and very uncomfortable for urology patients. Patients are typically instructed to arrive with a full bladder prior to uroflowmetry testing. This discomfort is particularly exacerbated in the urologic patient population, who typically have significant lower urinary tract issues such as urinary frequency and urgency.

Uroflowmetry systems are typically placed in unfamiliar voiding environments (i.e. not a bathroom), and do not have the ergonomics of a conventional toilet. These factors may result in patients' physical discomfort and mental anxiety during uroflowmetry tests.

### 3. Lack of Data Reliability

The International Continence Society (ICS) has several recommendations on good uroflowmetry practice to improve the quality of testing and data. In particular, uroflow should be conducted with patients in a preferred voiding position, with a “normal” desire to void, and with minimal anxiety and physical discomfort. That is, uroflowmetry should be representative of a normal voiding experience. As mentioned above, many current uroflowmetry systems do not have the ergonomics of a normal toilet, cannot provide a normal voiding experience, and this can result in less reliable or accurate measurements.

All of the above-mentioned factors causing patient discomfort, including increased urinary urgency due to waits, physical discomfort, and mental anxiety can compromise the capture of accurate and representative voiding data.

It would be beneficial to provide a uroflowmetry system which can help to avoid or reduce clinician workflow inefficiencies, patient discomfort and the lack of data reliability.

### SUMMARY OF THE INVENTION

In one aspect, the present invention provides a uroflowmetry system comprising: a receptacle for receiving urine; a plumbing unit for directing urine from the receptacle; flow-through fluid sensors for measuring urine flow; an output port from which urine exits the system; a data acquisition unit for receiving and collecting urine flow data from the flow-through fluid sensors; and a data processing and storage unit for processing and storing urine flow data received from the data acquisition unit.

In another aspect, the present invention provides a method of conducting a uroflowmetry test on a subject, comprising the steps of: obtaining a uroflowmetry system; having the subject direct a urine flow into the uroflowmetry system; and having the uroflowmetry system measure the urine flow; wherein the uroflowmetry system comprises: a receptacle for receiving urine; a plumbing unit for directing urine from the receptacle;

flow-through fluid sensors for measuring the urine flow; an output port from which urine exits the system; a data acquisition unit for receiving and collecting urine flow data from the flow-through fluid sensors; and a data processing and storage unit for processing and storing urine flow data received from the data acquisition unit.

### DESCRIPTION OF THE DRAWINGS

FIG. 1 illustrates a front view of a preferred embodiment of the system of the present invention.

FIG. 2 illustrates a perspective view of a preferred embodiment of the system of the present invention when mounted to a conventional toilet.

FIG. 3 illustrates a top view of a preferred embodiment of the system of the present invention when mounted to a conventional toilet.

FIG. 4 illustrates a perspective view of the main body of a preferred embodiment of the system of the present invention.

FIG. 5 illustrates a front view of the main body of a preferred embodiment of the system of the present invention.

FIG. 6 illustrates a front view of the main body of a preferred embodiment of the system of the present invention, with internal components shown in broken lines.

FIG. 7 illustrates a top view of the main body of a preferred embodiment of the system of the present invention.

FIG. 8 illustrates a preferred data acquisition unit that can be used with the system of the present invention.

FIG. 9 illustrates a preferred data processing and storage unit that can be used with the system of the present invention.

FIG. 10 illustrates a perspective view of a preferred embodiment of the system of the present invention where the data acquisition unit is part of the main body.

FIG. 11 illustrates a front view of a preferred embodiment of the system of the present invention where the data acquisition unit is part of the main body.

FIG. 12 illustrates a front view of a preferred flow redirection unit to be used in a preferred embodiment of the system of the present invention.

FIGS. 13A and 13B illustrate a cross section of a receptacle of a preferred embodiment of the system of the present invention, wherein a flow redirection unit is placed inside the receptacle.

### DESCRIPTION OF PREFERRED EMBODIMENTS

Two principles which were taken into account when designing the preferred uroflowmetry system of the present invention include: (A) toilet adapter; and (B) flow-through uroflowmetry.

### A. Toilet Adapter

The preferred system comprises a universal adapter for mounting to any standard toilet rim. By attaching the preferred system to a conventional toilet, patients can undergo uroflowmetry tests while voiding normally in any regular bathroom.

### B. Flow-Through Uroflowmetry

The preferred system measures relevant voiding metrics while urine passes directly into, through and out of the system (and preferably into a toilet).

Uroflowmetry testing is performed while patients void normally into a conventional toilet equipped with the preferred system. Voiding metrics are measured in real-time while urine passes through the system and into the toilet. After each test, urine is simply flushed away, and the system is immediately ready for the next patient. This flow-through eliminates or reduces the need for frequent manual emptying and cleaning of the system, and patients are able to void in a more familiar, comfortable environment.

The uroflowmetry system of the present invention preferably comprises the following preferred components:
1. Receptacle;
2. Flow redirection unit;
3. Plumbing unit;
4. Flow-through fluid sensors;
5. Output port;
6. Data acquisition unit;
7. Data processing and storage unit;
8. Data output unit; and
9. Toilet adapter unit.

The preferred components of the uroflowmetry system of the present invention and their roles are described in further detail below.

### 1. Receptacle

A receptacle comprising an input funnel collects voided urine from the patient and directs it to the plumbing unit and flow-through fluid sensors. Preferably, the funnel may be shaped like a hollow cone with a tube extending therefrom. Preferably, the funnel is wide at the top in order to efficiently capture and direct a downward flow of urine to the next component in the system. In another preferred embodiment, the receptacle is removable and replaceable from the system. For example, after a first patient uses the system, the first used receptacle can be easily and quickly removed and replaced with a second new and clean receptacle before the second patient uses the system.

### 2. Flow Redirection Unit

Voided urine entering the receptacle is typically directed to the plumbing unit in a smooth and laminar fashion by the receptacle's cone shaped walls. However, in instances where the voided stream is directed at the apex of the cone, turbulent flow may pass to the plumbing unit and affect the quality of recorded data. A flow redirection unit is preferably a dome placed in approximately the lower portion of the receptacle, preferably in the lower quarter, and redirects and redistributes streams aimed at the center of the receptacle towards its side walls. The dome preferably sits atop four short, evenly spaced rectangular supports to allow passage of urine below the dome and out of the receptacle.

### 3. Plumbing Unit

The plumbing unit preferably comprises a series of internal tubing that facilitate smooth/laminar flow of urine into the fluid sensors. The plumbing unit then guides the urine flow from the fluid sensors to the output port.

### 4. Flow-Through Fluid Sensors

Preferably, the system of the present invention comprises solid state MEMS (micro-electro-mechanical system) liquid flow sensors. As urine passes through these sensors, flow is detected and measured using a thermal mass-flow principle. These sensors have a fast response time and increased accuracy compared to other flow sensor types, and have no moving/mechanical components. This facilitates easy cleaning and eliminating points of failure such as mechanical wear-down and obstruction.

### 5. Output Port

Voided urine exits the system via an output port which extends from the plumbing unit. Preferably, voided urine passes from the system directly into a toilet bowl.

### 6. Data Acquisition Unit

In one preferred embodiment, the system of the present invention comprises a data acquisition unit such as an electronic microcontroller connected via analog signal to the fluid sensors. The microcontroller continuously samples analog data from the fluid sensors, preferably at a rate of 50 Hz or higher. When the analog signal is over a predetermined threshold (eg. “FLOW_THRESH”), the microcontroller begins submitting, via Serial protocol, a “START” signal and this analog data to a secondary data processing and storage unit. When the analog signal is below this threshold for a predetermined length of time (eg. “POST_VOID_TIME”), the current uroflowmetry test is declared complete, and the microcontroller submits a “STOP” signal to the Serial buffer. The data acquisition unit can either be part of the main body of the system or separate from the main body. In another preferred embodiment, a similar data acquisition unit achieves the same process with digital, rather than analog, data protocols.

### 7. Data Processing and Storage Unit

In one preferred embodiment, the system of the present invention comprises a miniaturized computer running custom Python software. This data processing and storage unit continuously reads data from the data acquisition unit via a two-way Serial buffer. Data between every “START” and “STOP” signal is saved as a single void, and a series of transformations, filters and analyses are applied to this data to process and output clinically relevant metrics. This information is preferably saved in on-board storage and also preferably submitted to the data output unit.

### 8. Data Output Unit

Preferably, the system of the present invention comprises a wired or wireless enabled printer that prints the processed voiding data in an organized, clinically useful format, which can include: 1) Volume vs. Time curve; 2) Flowrate vs. Time curve; 3) Maximum flowrate; 4) Average flowrate; 5) Time to maximum flowrate; 6) Total voiding time; 7) Total voided volume; and 8) Flowrate nomograms

### 9. Toilet Adapter Unit

Preferably, the system of the present invention comprises a universal mount that enables the entire system to be mounted to any toilet bowl. In one preferred embodiment, the toilet adapter unit is adjustable to allow attachment to toilets of different sizes.

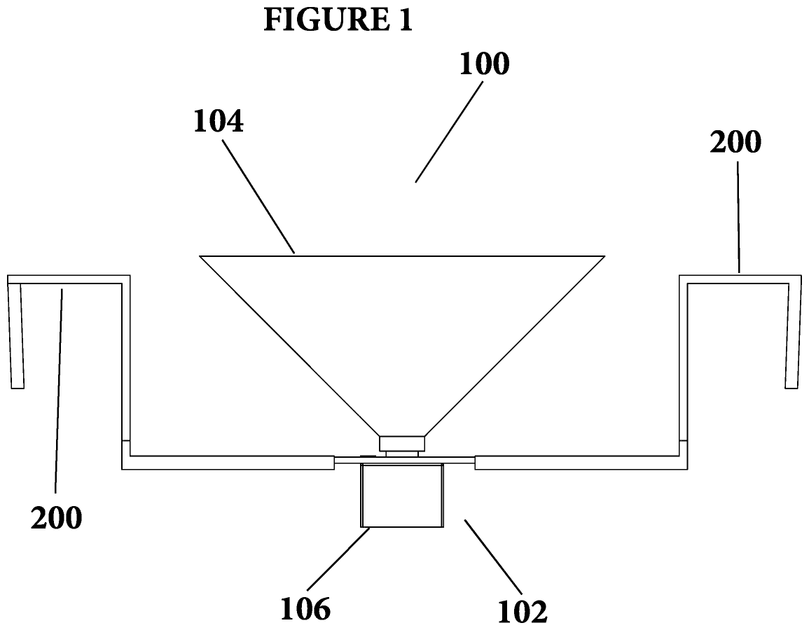

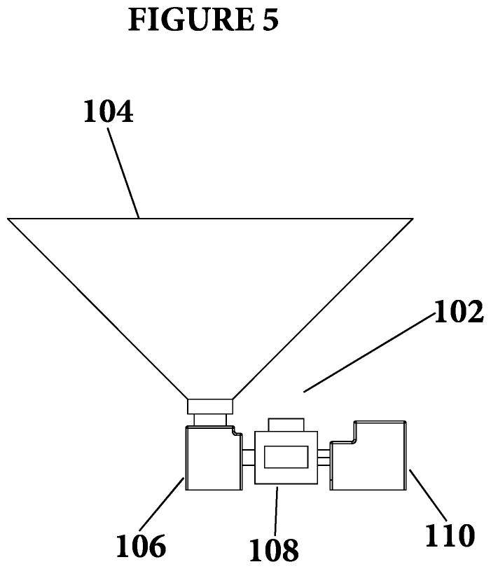

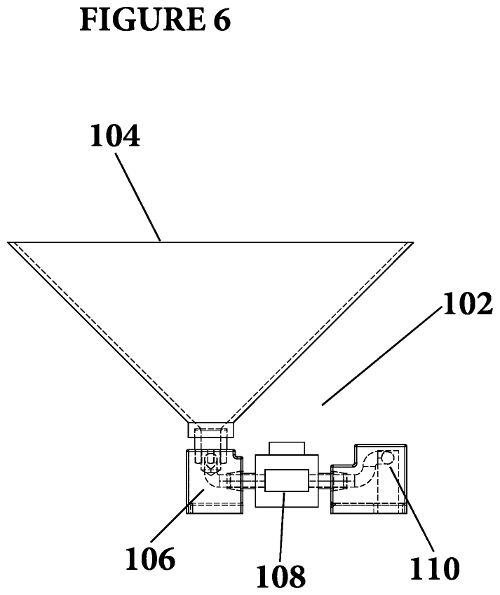

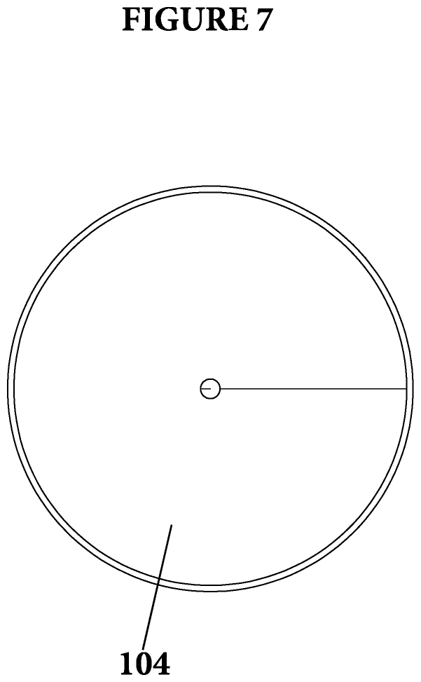

Referring to FIGS. 1 to 7, a preferred embodiment of the uroflowmetry system 100 of the present invention is shown. The system 100 is designed for use with a conventional toilet 500.

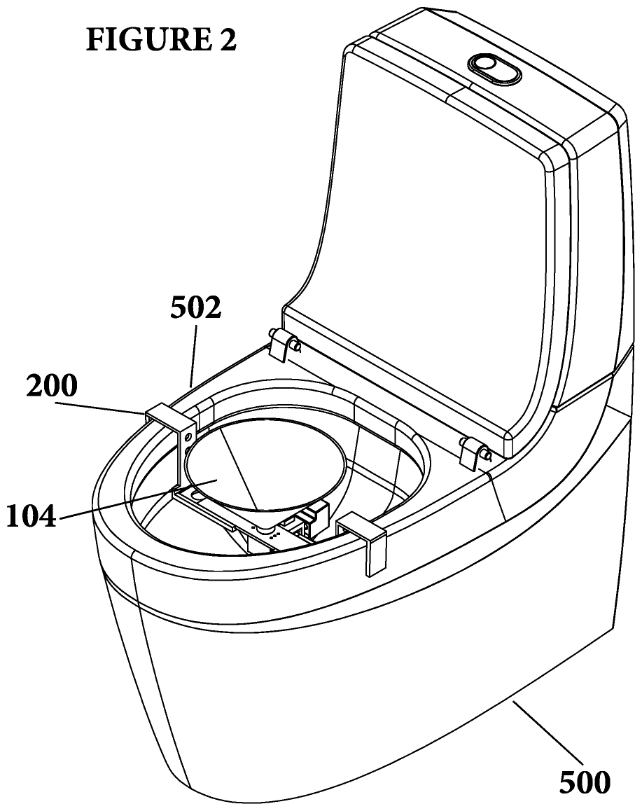

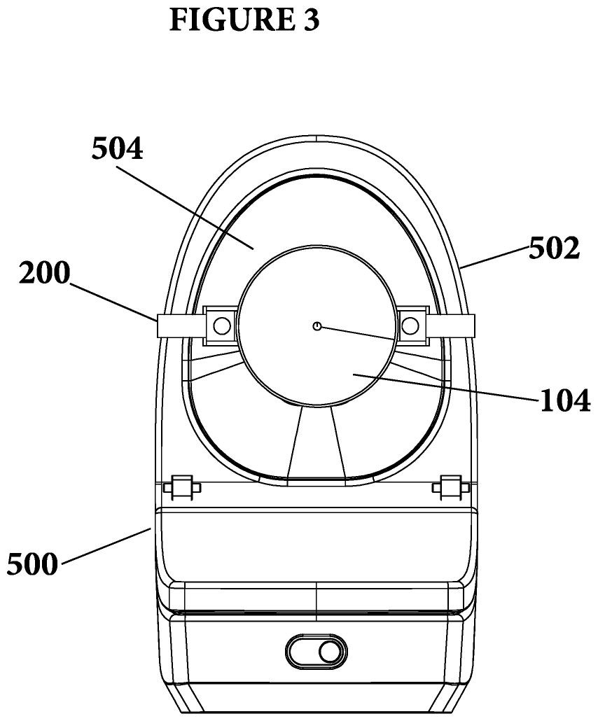

In this preferred embodiment, the system 100 has a main body 102 and a pair of brackets 200. The pair of brackets 200 facilitates mounting the system 100 to the rim 502 of the toilet 500, as shown in FIGS. 2 and 3. Preferably, as can be seen in FIGS. 1 to 3, the brackets 200 are U-shaped for fitting over the rim 502 of the toilet 500. Other types of brackets 200 may be used for this purpose as well.

The length between the main body 102 of the system 100 and the brackets 200 may be adjustable. For example, there may be a telescoping rod attaching the brackets 200 to the main body 102 of the system 100. By adjusting the length between the main body 102 and the brackets 200, the system 100 can be mounted to and used with toilets 500 of many different sizes.

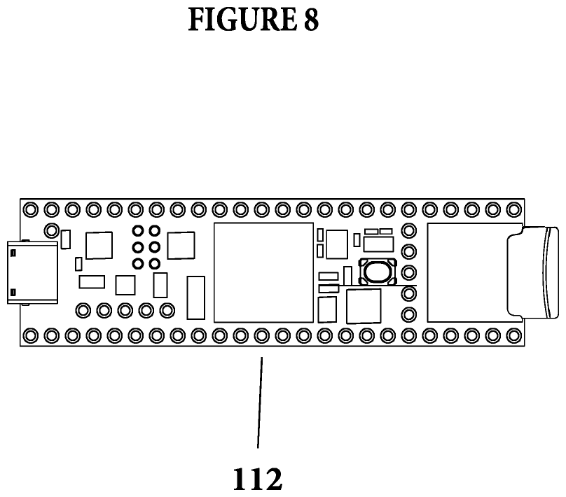

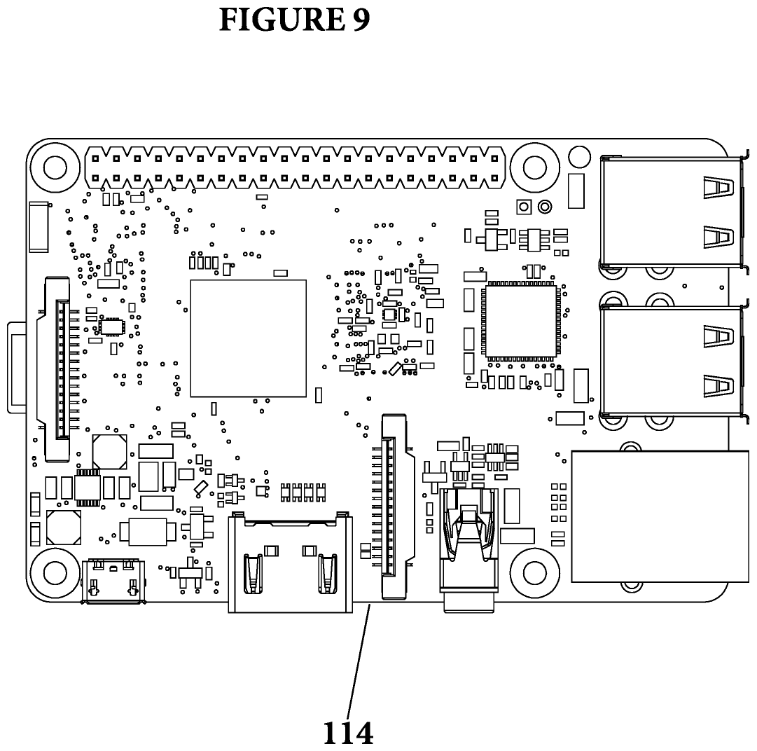

The main body 102 of the system 100 preferably comprises a receptacle 104, a plumbing unit 106, flow-through fluid sensors 108 and an output port 110. A data acquisition unit 112, as illustrated in FIG. 8, works with the flow-through sensors 108. As shown in FIG. 9, the system preferably also comprises a data processing and storage unit 114.

The receptacle 104 collects urine from the patient and carries out the input function of the system 100. In the preferred embodiment, the receptacle 104 is a funnel shaped like a hollow cone with a tube extending from the bottom of the cone. The wide top of the cone-shaped receptacle 104 makes it more likely that the system 100 will collect as much of the patient's urine as possible, with as little as possible spilling outside the system 100. The receptacle 104 may preferably be made of plastic or metal. When a patient voids, he or she directs their urine downwards into the receptacle 104. The cone-shaped receptacle 104 directs the collected urine downwards to the plumbing unit 106 of the system 100.

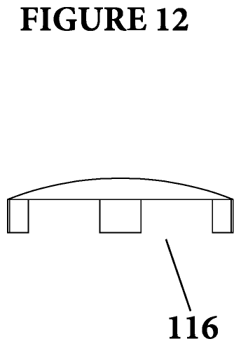

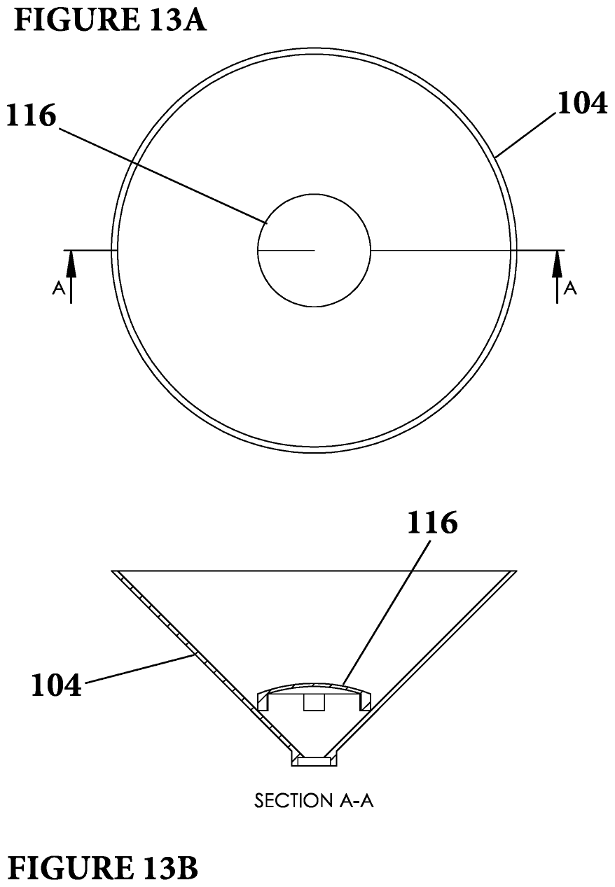

The preferred embodiment also comprises a flow redirection unit 116 as shown in FIGS. 12, 13A and 13B. The flow redirection unit 116 is preferably a dome shape structure placed in approximately the lower portion of the receptacle 104, preferably in the lower quarter. The flow redirection unit 116 helps redirect and redistribute urinary streams aimed at the center of the receptacle 104 away and onto its side walls. The dome preferably sits atop four short, evenly spaced rectangular supports to allow passage of urine below the dome and out of the receptacle 104. The plumbing unit 106 preferably comprises tubing which directs urine away from the receptacle 104 and into the flow-through fluid sensors 108. The tubing of the plumbing unit 106 then guides the urine from the fluid sensors 108 to the output port 110. The tubing of the plumbing unit 106 may preferably be made of plastic or metal.

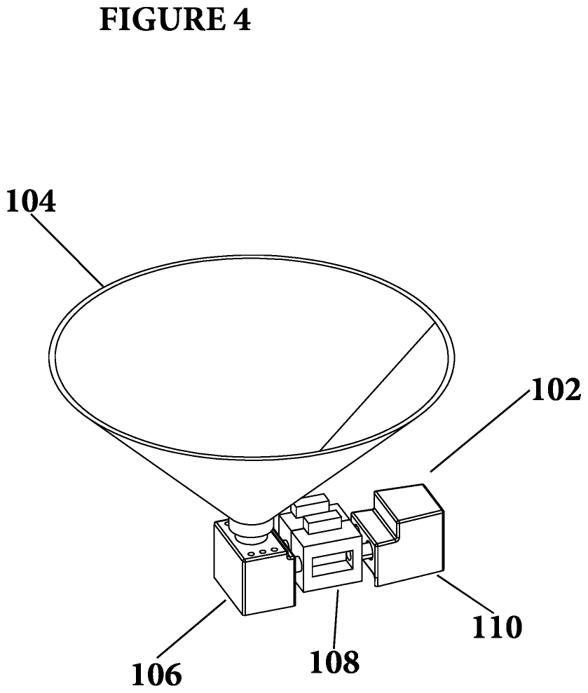

In the preferred embodiment of FIGS. 4 to 7, the flow-through fluid sensors 108 are solid state MEMS (micro-electro-mechanical system) liquid flow sensors. Other types of sensors may be used in the system 100 of the present invention, as long as they allow urine to flow through the plumbing unit 106 and they detect and measure the flow. As urine passes through these flow-through fluid sensors 108, flow is detected and measured using a thermal mass-flow principle. In other embodiments, flow may be detected via other techniques, as long as they permit accurate measurement of fluid flowrate, such as capacitive sensors, magnetic induction, cantilever, air displacement, water level sensors, paddlewheel, rotameter, or optical.

An output port 110 extends from the flow-through sensors 108. After urine passes over the flow-through fluid sensors 108, and measurements of the flow are detected and measured, urine exits the system 100 from the output port 110. The output port 110 may be in the form of a simple hole or a tube which directs the urine in a desired direction out the system 100. If the system 100 is mounted to a toilet 500, then the voided urine passes out the output port and into the bowl 504 of the toilet 500. The output port 110 may preferably be made of plastic or metal.

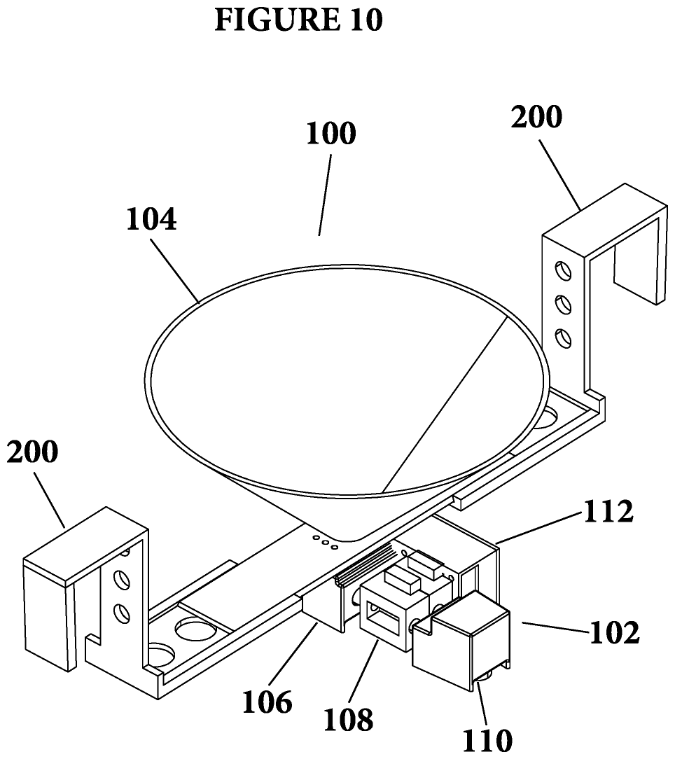

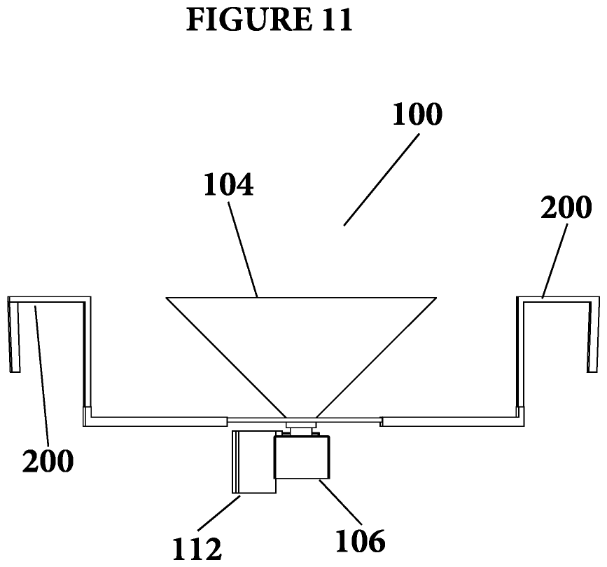

In one preferred embodiment, as shown in FIGS. 10 and 11, the main body 102 of the system 100 further comprises a data acquisition unit 112 which collects data from the flow-through fluid sensors 108. In another embodiment, as shown in FIGS. 1 to 7, the data acquisition unit 112 is separate from the main body 102 of the system 100.

In the preferred embodiment shown in FIGS. 10 and 11, there is a data acquisition unit 112 connected via analog signal to the flow-through fluid sensors 108. The data acquisition unit 112 continuously samples analog data from the fluid sensors 108, preferably at a minimum 50 Hz rate. When the analog signal is over a predetermined threshold (eg. “FLOW_THRESH”), the data acquisition unit 112 begins submitting, via Serial protocol, a “START” signal and this analog data to a secondary data processing and storage unit 114. When the analog signal is below the threshold for a predetermined length of time (i.e. “POST_VOID_TIME”), the current uroflowmetry test is declared complete, and the data acquisition unit 112 submits a “STOP” signal to the Serial buffer. In other embodiments, the data acquisition unit may achieve this goal via digital data and signal protocols.

Preferably, the system 100 further comprises a data processing and storage unit 114, such as that shown in FIG. 9, for processing and storing urine flow data received from the data acquisition unit 112. In a preferred embodiment, the data processing and storage unit 114 is located in the main body 102 of the system 100, while in another embodiment, the data processing and storage unit 114 is located outside the main body 102 of the system 100.

The data processing and storage unit 114 may preferably be a miniaturized computer running custom Python software. This data processing and storage unit 114 continuously reads data from the data acquisition unit 112 via a two-way Serial buffer. Data between every “START” and “STOP” signal is saved as a single void, and a series of transformations, filters and analyses are applied to this data to process and output clinically relevant metrics. This information is preferably saved in on-board storage and also preferably submitted to a data output unit.

Preferably, the system 100 of the present invention comprises a data output unit which converts processed urine flow data from the data processing and storage unit 114 to a readable format. The data output unit may preferably comprise a wired or wireless enabled printer that prints the processed voiding data in an organized, clinically useful format.

The system of the present invention provides at least the following benefits.

### 1. More Efficient Clinician Workflow

The system of the present invention can be attached to a conventional toilet and permits flow-through. Therefore, uroflowmetry can be performed entirely by the patient without additional interventions, emptying or cleaning by the clinician or other staff. The clinician can focus on patient care rather than constantly maintaining a piece of clinical equipment.

### 2. Increased Patient Comfort

Since the system of the present invention does not require cleaning/maintenance between each use, wait-times between tests are dramatically decreased. This reduces patient discomfort and anxiety associated with waiting to void while having a full bladder, especially for patients with moderate to severe lower urinary tract symptoms, such as those patients suffering from symptoms of overactivity, urgency, and urge related incontinence.

The system of the present invention allows patients to undergo uroflowmetry testing by simply voiding into a conventional toilet as they normally would. This facilitates, as close as possible, a normal voiding environment, position, and, therefore, optimizes the patient's physical comfort and decreases mental anxiety.

### 3. Increased Data Reliability

The system of the present invention helps to optimize patients' voiding position, their “normal” desire to void, decreases anxieties and increases physical comfort, per International Continence Society recommendations. By optimizing these factors, the system of the present invention facilitates the capture of more representative and accurate voiding data.

Any of the software applications disclosed herein can be used separately or can be combined with others in any combination. Any of the applications described herein can include machine-readable instructions for execution by: (a) a processor, (b) a controller, and/or (c) any other suitable processing device. Any application, algorithm or software disclosed herein can be embodied in software stored on a non-transitory tangible medium such as, for example, a flash memory, a CD-ROM, a floppy disk, a hard drive, a digital versatile disk (DVD), or other memory devices, but persons of ordinary skill in the art will readily appreciate that the entire application and/or parts thereof could alternatively be executed by a device other than a controller and/or embodied in firmware or dedicated hardware in a well-known manner (e.g., it may be implemented by an application specific integrated circuit (ASIC), a programmable logic device (PLD), a field programmable logic device (FPLD), discrete logic, etc.). Further, persons of ordinary skill in the art will readily appreciate that many other methods of implementing the example machine readable instructions may alternatively be used.

The scope of the claims should not be limited by the preferred embodiments set forth in the examples, but should be given the broadest interpretation consistent with the description as a whole.

1. A uroflowmetry system comprising:
a receptacle for receiving urine;
a plumbing unit for directing urine from the receptacle;
flow-through fluid sensors for measuring urine flow;
an output port from which urine exits the system;
a data acquisition unit for receiving and collecting urine flow data from the flow-through fluid sensors; and
a data processing and storage unit for processing and storing urine flow data received from the data acquisition unit.

2. The uroflowmetry system of claim 1, further comprising:
a data output unit which converts processed urine flow data from the data processing and storage unit to a readable format.

3. The uroflowmetry system of claim 1, further comprising:
a toilet adapter unit for mounting the system to a rim of a toilet bowl.

4. The uroflowmetry system of claim 1, wherein the receptacle comprises a funnel;
and the system further comprises a flow redirection unit placed inside the receptacle, wherein the flow redirection unit is a structure which redirects urinary streams aimed at a center portion of the receptable to side walls of the receptacle.

5. The uroflowmetry system of claim 1, wherein the plumbing unit comprises tubing which receives urine from the receptacle, and directs urine to the flow-through fluid sensors and the output port.

6. The uroflowmetry system of claim 1, wherein the flow-through fluid sensors comprise micro-electro-mechanical system (MEMS) liquid flow sensors.

7. The uroflowmetry system of claim 1, wherein the data acquisition unit, upon receiving urine flow data from the flow-through fluid sensors that the urine flow has gone above a predetermined threshold, begins sending the urine flow data to the data processing and storage unit.

8. The uroflowmetry system of claim 7, wherein the data acquisition unit, upon receiving urine flow data from the flow-through fluid sensors that the urine flow has gone below a predetermined threshold, stops sending the urine flow data to the data processing and storage unit.

9. The uroflowmetry system of claim 1, wherein the data processing and storage unit processes the urine flow data received from the data acquisition unit into clinically relevant metrics.

10. The uroflowmetry system of claim 3, wherein the toilet adapter unit is adjustable to allow mounting to toilets of different sizes.

11. A method of conducting a uroflowmetry test on a subject, comprising the steps of:
obtaining a uroflowmetry system;
having the subject direct a urine flow into the uroflowmetry system; and
having the uroflowmetry system measure the urine flow;
wherein the uroflowmetry system comprises:
a receptacle for receiving urine;
a plumbing unit for directing urine from the receptacle;
flow-through fluid sensors for measuring the urine flow;
an output port from which urine exits the system;
a data acquisition unit for receiving and collecting urine flow data from the flow-through fluid sensors; and
a data processing and storage unit for processing and storing urine flow data received from the data acquisition unit.

12. The method of claim 11, said uroflowmetry system further comprising:
a data output unit which converts processed urine flow data from the data processing and storage unit to a readable format.

13. The method of claim 11, said uroflowmetry system further comprising:
a toilet adapter unit for mounting the system to a rim of a toilet bowl.

14. The method of claim 11, wherein the receptacle comprises a funnel;
and the system further comprises a flow redirection unit placed inside the receptacle, wherein the flow redirection unit is a structure which redirects urinary streams aimed at a center portion of the receptable to side walls of the receptacle.

15. The method of claim 11, wherein the plumbing unit comprises tubing which receives urine from the receptacle, and directs urine to the flow-through fluid sensors and the output port.

16. The method of claim 11, wherein the flow-through fluid sensors comprise micro-electro-mechanical system (MEMS) liquid flow sensors.

17. The method of claim 11, wherein the data acquisition unit, upon receiving urine flow data from the flow-through fluid sensors that the urine flow has gone above a predetermined threshold, begins sending the urine flow data to the data processing and storage unit.

18. The method of claim 17, wherein the data acquisition unit, upon receiving urine flow data from the flow-through fluid sensors that the urine flow has gone below a predetermined threshold, stops sending the urine flow data to the data processing and storage unit.

19. The method of claim 11, wherein the data processing and storage unit processes the urine flow data received from the data acquisition unit into clinically relevant metrics.

20. The method of claim 13, wherein the toilet adapter unit is adjustable to allow mounting to toilets of different sizes.
